SEQUENCE LISTING (14 sequences)

SEQ ID NO: 1 - DNA, 20 bp, ARTIFICIAL SEQUENCE
  SYNTHETIC PRIMER
cgaccatctgccgctttgag

SEQ ID NO: 2 - DNA, 20 bp, ARTIFICIAL SEQUENCE
  SYNTHETIC PRIMER
ccccctgtcccccattccta

SEQ ID NO: 3 - DNA, 25 bp, ARTIFICIAL SEQUENCE
  SYNTHETIC PRIMER
cctcctccatggatctgcttattca

SEQ ID NO: 4 - DNA, 26 bp, ARTIFICIAL SEQUENCE
  SYNTHETIC PRIMER
caggtcttcacctgtttgtagctgag

SEQ ID NO: 5 - DNA, 23 bp, ARTIFICIAL SEQUENCE
  SYNTHETIC PRIMER
gcgtacgcaaattaaagtccaga

SEQ ID NO: 6 - DNA, 25 bp, ARTIFICIAL SEQUENCE
  SYNTHETIC PRIMER
cagcatcctaaacagctcgcagaat

SEQ ID NO: 7 - DNA, 24 bp, ARTIFICIAL SEQUENCE
  SYNTHETIC PRIMER
gcccgcttctcttacagtgtgatt

SEQ ID NO: 8 - DNA, 25 bp, ARTIFICIAL SEQUENCE
  SYNTHETIC PRIMER
agtacgtgcagacggtggtagttct

SEQ ID NO: 9 - DNA, 18 bp, ARTIFICIAL SEQUENCE
  SYNTHETIC PRIMER
cccccggcggcaatagca

SEQ ID NO: 10 - DNA, 20 bp, ARTIFICIAL SEQUENCE
  SYNTHETIC PRIMER
tcggcgccggggagatacat

SEQ ID NO: 11 - DNA, 20 bp, ARTIFICIAL SEQUENCE
  SYNTHETIC PRIMER
agctatgcccggacctccat

SEQ ID NO: 12 - DNA, 20 bp, ARTIFICIAL SEQUENCE
  SYNTHETIC PRIMER
gcctgcagcaggaggatctt

SEQ ID NO: 13 - DNA, 20 bp, ARTIFICIAL SEQUENCE
  SYNTHETIC PRIMER
gctcgtcgtcgacaacggct

SEQ ID NO: 14 - DNA, 25 bp, ARTIFICIAL SEQUENCE
  SYNTHETIC PRIMER
caaacatgatctgggtcatcttctc

(19) United States
(12) Patent Application Publication
(10) Pub. No.: US 2010/0105132 A1
(43) Pub. Date: Apr. 29, 2010

(54) Human Mesenchymal stem cells and preparation thereof

(75) Inventors: Satish Mahadeorao Totey, Bangalore (IN); Kumar Uday Kolkundkar, Bangalore (IN); Rakhi Pal, Bangalore (IN); Madhuri Hanwate, Bangalore (IN)

Correspondence Address: SCHWEGMAN, LUNDBERG & WOESSNER, P.A., P.O. BOX 2938, MINNEAPOLIS, MN, 55402, US

(73) Assignee: Stempeutics Research Private Limited, Bangalore, IN (US)

(21) Appl. No.: 12/589,252

(22) Filed: Oct. 20, 2009

Related U.S. Application Data
(63) Continuation of application No. PCT/IN2008/000255, filed on Apr. 28, 2008.

(30) Foreign Application Priority Data
Apr. 28, 2007 (IN) 861/CHE/2007

(51) Int. Cl. C12N 5/0735; C12N 5/02

(52) U.S. Cl. 435/352; 435/366; 435/405

(57) ABSTRACT

The present invention provides a process of isolation, proliferation and/or maintenance of mesenchymal stem cells (MSCs). The invention further provides a culture medium for proliferation and/or maintenance of human mesenchymal stem cells in xeno-free conditions. The culture medium provided in the present invention proliferates and/or maintains mesenchymal stem cell expansion while maintaining a multipotent phenotype.

### PRIORITY CLAIM TO RELATED APPLICATIONS

This application is a national stage application under 35 U.S.C. §371 of PCT/IN2008/000255, filed Apr. 23, 2008, and published as WO 2008/129563 A2 on Oct. 30, 2008, which claims priority to Indian Application No. 861/CHE/2007, filed Apr. 23, 2007, which applications and publication are incorporated herein by reference and made a part hereof in their entirety, and the benefit of priority is claimed thereto.

### FIELD OF INVENTION

The present invention relates to a process of isolation, proliferation and/or maintenance of mesenchymal stem cells (MSCs). The invention further provides a culture medium for proliferation and/or maintenance of human mesenchymal stem cells in xeno-free conditions.

### BACKGROUND OF INVENTION

One of the biggest challenges for the biomedical research lies in the development of therapeutics strategies which allow to replaced or repair cells or tissues damaged or destroyed in the most devastating or disabling diseases.

One of the most promising therapeutic strategies is the cell therapy, founded in the well-known fact that it is possible to have available cells, more or less undifferentiated, which are able to divide themselves generating functional differentiated cells and, in some cases, can even regenerate themselves. Among these are included the stem cells. These cells can be found in the embryo and even in adult tissues. Cell therapy consists, therefore, in the transplantation or implant in the patient of the sufficient quantity of stem cells to repair and restore the functionality of a damaged organ.

Mesenchymal stem cells (MSCs) are present in different types of adult tissues such as bone marrow, limbal cells, adipose tissue etc and constitute a population of cells that can be isolated, expanded in culture, and characterized in vitro and in vivo (Pittenger and Martin (2004) Circ. Res. 95:9-20). MSCs are able to differentiate into multiple cell lineages, including osteoblasts, chondrocytes, endothelial cells, and neuronal cells, (Kassem et al. (2004) Basic Clin. Pharmacol. Toxicol. 95:209-214; Pittenger and Martin (2004) Circ. Res. 95:9-20). In recent years, MSCs have generated a high level of experimental and clinical interest due to their potential for a range of therapeutic uses including repair of damaged or diseased tissues (Baksh et al. (2004) J. Cell. Mol. Med. 8:301-316; Barry and Murphy (2004) Int. J. Biochem. Cell Bio 36:568-584). Mesenchymal stem cells are difficult to grow without serum because they detach and die in culture. These MSCs can be maintained in an attached state in vitro with minimal serum (e.g., <1%), although such an environment provides little stimulation for MSCs to proliferate and grow. Earlier serum-free cell culture environments have been reported for MSC expansion (Lennon et al. (1995) Exp. Cell Res. 219:211-222; U.S. Pat. No. 5,908,782) however, it is difficult to collect sufficient amount of human serum for this purpose since large amount of serum is required for mesenchymal stem cell to grow.

It is therefore desirable to provide xeno-free culture conditions for expansion of mesenchymal stem cells in a way that they grow for several passages without loosing its functional characteristics and have great utility in the field of cellular therapy.

The creation of highly defined environments for cell expansion is of great importance for quality purposes, and serum levels are typically very ill-defined (U.S. Pat. No. 5,908,782). In addition, there is a risk of Bovine Spongiform Encephalopathy (BSE) contamination in patients receiving cells cultured in the presence of serum.

### SUMMARY OF THE INVENTION

The present invention provides a process of isolation, proliferation and/or maintenance of mesenchymal stem cells (MSCs), wherein the MSCs can be isolated from various samples such as bone marrow, corneal epithelial tissue and adipose tissue. The invention further provides a culture medium for proliferation and/or maintenance of human mesenchymal stem cells in xeno-free conditions.

One aspect of the present invention is to provide a process of isolation of mesenchymal stem cells (MSC) from a sample, said process comprising;
treating said sample with an antibody against an antigen, wherein said antigen is selected from a group consisting of CD44, CD50, CD54, CD73, CD90, CD105, CD106, CD117, oct 3/4, Actin filament associated protein (AFAP), Frizzled7 (FZD7), Dickkopf3 (DKK3), Protein tyrosine phosphatase receptor F (PTPRF), RAB3B, and Stro-1;
obtaining cells expressing said antigen; wherein said cells are mesenchymal stem cells; and
culturing said mesenchymal stem cells in a growth medium selected from the group consisting of i) a culture medium comprising a basal medium selected from the group consisting of KO-DMEM, DMEM-LG and DMEM-F12 (1:1) or a combination thereof; 5-10% fetal bovine serum (FBS), growth factors, 200 mM GlutaMax™ and antibiotics; ii) a culture medium comprising basal medium selected from the group consisting of KO-DMEM, DMEM-LG and DMEM-F12 (1:1) or a combination thereof; growth factors, human serum, 200 mM GlutaMax™ and antibiotics; iii) a culture medium comprising basal medium selected from the group consisting of KO-DMEM, DMEM-LG and DMEM-F12 (1:1) or a combination thereof; growth factors, human plasma, heparin, 200 mM GlutaMax™ and antibiotics; and iv) a culture medium comprising basal medium selected from the group consisting of KO-DMEM, DMEM-LG and DMEM-F12 (1:1) or a combination thereof; growth factors, human plasma or human serum or a combination thereof, heparin, 200 mM GlutaMax™ and antibiotics.

Another aspect of the present invention relates to a cell culture medium for proliferation and/or maintenance of mesenchymal stem cells (MSCs), wherein said culture medium comprising KO-DMEM; growth factors selected from the group consisting of 5 to 100 ng/ml platelet derived growth factor (PDGF), 5 to 50 ng/ml transforming growth factor-beta (TGF-beta), 4 to 100 ng/ml keratinocyte growth factor (KGF), 4 to 80 ng/ml basic Fibroblast growth factor (bFGF), 5 to 50 ng/ml Epidermal growth factor (EGF); 10 to 100000 U/ml Leukemia Inhibitory growth factor (LIF) and 5 to 50 ng/ml Insulin-like growth factor-I (IGF-I); 5 to 10% (w/w) fetal bovine serum (FBS); 200 mM GlutaMax™; and antibiotics.

Yet another aspect of the present invention relates to a cell culture medium for proliferation and/or maintenance of mesenchymal stem cells (MSCs), wherein said culture medium comprising KO-DMEM; growth factors selected from the group consisting of 5 to 100 ng/ml platelet derived growth factor (PDGF), 5 to 50 ng/ml transforming growth factor-beta (TGF-beta), 4 to 100 ng/ml keratinocyte growth factor (KGF), 4 to 80 ng/ml basic Fibroblast growth factor (bFGF), 5 to 50 ng/ml Epidermal growth factor (EGF); 10 to 100000 U/ml Leukemia Inhibitory growth factor (LIF) and 5 to 50 ng/ml Insulin-like growth factor-I (IGF-I); human plasma or human serum or a combination thereof; 200 mM GlutaMax™; and antibiotics.

Still yet another aspect of the present invention relates to a cell culture medium for proliferation and/or maintenance of mesenchymal stem cells (MSCs), wherein said culture medium comprising basal medium selected from the group consisting of KO-DMEM, DMEM-LG and DMEM-F12 (1:1) or a combination thereof; growth factors selected from the group consisting of 5 to 100 ng/ml platelet derived growth factor (PDGF), 5 to 50 ng/ml transforming growth factor-beta (TGF-beta), 4 to 100 ng/ml keratinocyte growth factor (KGF), 4 to 80 ng/ml basic Fibroblast growth factor (bFGF), 5 to 50 ng/ml Epidermal growth factor (EGF); 10 to 100000 U/ml Leukemia Inhibitory growth factor (LIF) and 5 to 50 ng/ml Insulin-like growth factor-I (IGF-I); 0.5 to 1% human serum; 200 mM GlutaMax™; and antibiotics.

Further aspect of the present invention relates to a cell culture medium for proliferation and/or maintenance of mesenchymal stem cells (MSCs), wherein said culture medium comprising basal medium selected from the group consisting of KO-DMEM, DMEM-LG and DMEM-F12 (1:1) or a combination thereof; growth factors selected from the group consisting of 5 to 100 ng/ml platelet derived growth factor (PDGF), 5 to 50 ng/ml transforming growth factor-beta (TGF-beta), 4 to 100 ng/ml keratinocyte growth factor (KGF), 4 to 80 ng/ml basic Fibroblast growth factor (bFGF), 5 to 50 ng/ml Epidermal growth factor (EGF); 10 to 100000 U/ml Leukemia Inhibitory growth factor (LIF) and 5 to 50 ng/ml Insulin-like growth factor-I (IGF-I); 1 to 20% human plasma; 200 mM GlutaMax™; 1000 to 10,000 U/ml heparin and antibiotics.

Another aspect of the present invention relates to a cell culture medium for proliferation and/or maintenance of mesenchymal stem cells (MSCs), wherein said culture medium comprising basal medium selected from the group consisting of KO-DMEM, DMEM-LG and DMEM-F12 (1:1) or a combination thereof; growth factors selected from the group consisting of 5 to 100 ng/ml platelet derived growth factor (PDGF), 5 to 50 ng/ml transforming growth factor-beta (TGF-beta), 4 to 100 ng/ml keratinocyte growth factor (KGF), 4 to 80 ng/ml basic Fibroblast growth factor (bFGF), 5 to 50 ng/ml Epidermal growth factor (EGF); 10 to 100000 U/ml Leukemia Inhibitory growth factor (LIF) and 5 to 50 ng/ml Insulin-like growth factor-I (IGF-I); 0.5 to 1% human serum or 1 to 20% human plasma; 200 mM GlutaMax™; 1000 to 10,000 U/ml heparin and antibiotics.

### BRIEF DESCRIPTION OF DRAWINGS

FIG. 1 shows phase contrast photomicrographs of a monolayer of adult bone marrow derived MSCs under xeno-free condition.

(a) MSCs cultured in 1% human serum

(b) MSCs cultured in 10% human plasma

FIG. 2 shows a series of photomicrographs of a monolayer of adult bone marrow derived MSCs differentiated into osteoblasts. (10×)

(a) Osteoblast differentiation at Passage 5 of adult bone marrow derived MSCs.

(i) MSCs cultured in DMEM-F12 followed by osteoblast differentiation

(ii) MSCs cultured in DMEM-KO followed by osteoblast differentiation

(b): Osteoblast differentiation at Passage 25 of adult bone marrow derived MSCs.

(i) MSCs cultured in DMEM-F12 followed by osteoblast differentiation

(ii) MSCs cultured in DMEM-KO followed by osteoblast differentiation

FIG. 3 shows a series of photomicrographs of a monolayer of adult bone marrow derived MSCs differentiated into adipocytes. (10×)

(a) Adipocyte differentiation at Passage 5 of adult bone marrow derived MSCs.

(i) MSCs cultured in DMEM-F12 followed by adipocyte differentiation

(ii) MSCs cultured in DMEM-KO followed by adipocyte differentiation

(b): Osteoblast differentiation at Passage 25 of adult bone marrow derived MSCs.

(i) MSCs cultured in DMEM-F12 followed by adipocyte differentiation

(ii) MSCs cultured in DMEM-KO followed by adipocyte differentiation

FIG. 4 illustrates the Karyotype of adult human bone marrow derived MSCs (100×).

(a): Karyotype at P25 of adult BM-MSCs cultured in DMEM-F12
(b): Karyotype at P25 of adult BM-MSCs cultured in DMEM-KO

### DETAILED DESCRIPTION OF THE INVENTION

The present invention provides a process of isolation, proliferation and/or maintenance of mesenchymal stem cells (MSCs), wherein the MSCs can be isolated from various samples such as bone marrow, corneal epithelial tissue and adipose tissue. The invention further provides a culture medium for proliferation and/or maintenance of human mesenchymal stem cells in xeno-free conditions. The culture medium provided in the present invention proliferates and/or maintains mesenchymal stem cell expansion while maintaining a multipotent phenotype. The invention also provides isolated mesenchymal stem cells.

In the present invention, the singular forms “a”, “an”, and “the” include plural reference, unless the context clearly dictates otherwise.

The term “Mesenchymal stem cells” or “MSCs” used herein is interchangeable.

In the present disclosure the term “xeno-free medium” refers to medium devoid of animal serum (for example, non-human serum).

The term “propagation” and “proliferation” used herein are interchangeable.

In one embodiment, the present disclosure relates to isolation and culture of mesenchymal stem cells from adult human bone marrow without using any animal products or material or animal derived growth supplements (for example, non-human products or material or non-human derived growth supplements).

The present invention provides improved method of isolation and methods for promoting mesenchymal stem cells (MSCs) expansion while maintaining the multipotent phenotype of these cells. Large scale production of MSCs under xeno-free cell culture system for MSCs expansion is provided. Isolation of MSCs from human adult bone marrow or corneal epithelial tissue comprises of improved method of separation of target cells using cocktail of antibodies. Large scale production of MSCs under xeno-free condition culture system comprises plating of MSCs in cell factory having total surface of 6360 square cm in a culture media that is supplemented with at least one or combination of human plasma or human serum or combination thereof. Cell culture system may also include growth promoting and self renewal factor which are suitable for MSCs expansion.

In the present invention, the culture environment supports long-term propagation of the mesenchymal stem cells. Culture environment described in this invention allows proliferation of stem cells useful in the production of important products for use in human therapy.

Methods of the present invention are directed to the use of these xeno-free cell culture systems to promote the expansion of MSCs. The expanded MSCs of the present invention can be used to treat various disorders or diseases, particularly those of the cardiovascular, neurodegenerative diseases, diabetes, autoimmune diseases, and bone, cartilage and muscle disorders.

The present invention relates to isolation and culture of mesenchymal stem cells from adult human bone marrow and corneal epithelial tissue without using any animal products or material or animal derived growth supplements.

This invention relates to isolation, culture and expansion of mesenchymal stem cells from adult human bone marrow and corneal epithelial tissue. More particularly, this invention relates to novel method of isolation and culture of mesenchymal stem cells in a defined media which is suitable for use in cell therapy including promoting angiogenesis in various tissues, autoimmune diseases, neurodegenerative diseases, cardiovascular diseases, cancer, inflammatory diseases and disorders, and promoting would healing.

The present invention provides an improved method of isolation of mesenchymal stem cells from adult human bone marrow and corneal epithelial tissue. Isolation process comprise of negative selection of unwanted cells using human surface antibodies.

The human antibodies comprises of cell surface markers such as CD3, CD4, CD8, CD14, CD19, CD38, and CD66b and glycophorin-A alone or combination thereof so as to remove unwanted cells of negative fraction such as hematopoietic cells. The antibody cocktail solution, 10 μl directly added to 10 ml of fresh bone marrow samples and incubated for 20 minutes at room temperature. The unwanted cells form the antigen-antibody complex. This makes the cells heavier and hence settles with the red cell population at the bottom layer. Thus, an enriched population of MSCs at the Ficoll interface was obtained. The target cells are collected as a purified population from Ficoll interface. Isolation process comprises of negative selection of unwanted cells using cocktail of antibodies. Antibodies may be of polyclonal or monoclonal in nature.

The present invention also provides an improved method of isolation of mesenchymal stem cells from corneal epithelial tissue. Isolation process comprise of positive selection of cells of interest. Here positive selection of cells of interest is mesenchymal stem cells.

The present invention provides the method of culturing isolated mesenchymal stem cells in large quantity in cell factory with total surface area is 6360 sq. cm.

Isolated target cells are cultured in a culture medium, which comprises of nutrient medium, human serum, human plasma, heparin and growth factors.

The present invention also provides the method of isolation of human serum from human plasma. Isolation process comprise of 10× recalcification (0.25M Cacl2.6H20-55 gm and 0.08M Mgcl2.6H20-16 gms) in 100 ml distilled water is prepared autoclaved at 121° C. for 20 min at 15 lbs pressure. 1.5 ml of recalciform solution is added to 1 unit of blood or 250 ml of plasma which has been brought to room temp. Incubation is carried out at 37° C. for 30-60 min (unit clot form) followed by overnight storage at 4° C. Centrifugation is carried out at 1500 rpm for 20 min. Serum is separated aseptically.

In one embodiment culture media comprises of either, DMEM-LG, KO-DMEM, DMEM-F 12 (1:1) or a combination thereof.

In another embodiment culture media is supplemented with either human serum or human plasma, or heparin treated human plasma or human serum obtained from healthy donors having blood group of “O” Rh positive, human serum albumin or combination thereof. In the present invention either 10% human plasma or 9% human plasma+1% human serum, or 9% heparin treated human plasma+1% human serum or 9% human plasma+5% human serum albumin or combination of thereof.

The present invention provides a media comprises of human serum is provided at a concentration of 0.5 to 5%.

The present invention provides a media comprises of human plasma is provided at a concentration of 1 to 15%.

The present invention provides a media comprises of heparin is provided at a concentration of 0.1 to 10,000 UL/ml.

In another embodiment culture media is supplemented with growth factors. Tissue culture medium further includes at least one growth factor or combination thereof.

The present invention provides the culture media comprises of the growth factors selected from group of platelet derived growth factor (PDGF), transforming growth factor-beta (TGF-beta), keratinocyte growth factor (KGF), basic Fibroblast growth factor (bFGF), Epidermal growth factor (EGF), Insulin-like growth factor-I (IGF-I), and Leukemia Inhibitory growth factor (LIF) or combination thereof.

The present invention still further features in the described preferred invention the bFGF is provided at a concentration of 4 to 80 ng/ml.

The present invention still further features in the described preferred invention the bFGF is provided at a concentration of 10 ng/ml.

The present invention still further features in the described preferred invention the PDGF is provided at a concentration of 5 to 100 ng/ml.

The present invention still further features in the described preferred invention the PDGF is provided at a concentration of 20 ng/ml.

The present invention still further features in the described preferred invention the EGF is provided at a concentration of 5 to 50 ng/ml.

The present invention still further features in the described preferred invention the EGF is provided at a concentration of 15 ng/ml.

The present invention still further features in the described preferred invention the LIF is provided at the concentration of 10 to 100000 U/ml.

The present invention still further features in the described preferred invention the LIF is provided at a concentration of 10,000 U/ml.

The present invention still further features in the described preferred invention the TGF is provided at the concentration of 5 to 50 ng/ml.

The present invention still further features in the described preferred invention the TGF is provided at the concentration of 20 ng/ml.

The present invention still further features in the described preferred invention the IGF is provided at the concentration of 5 to 50 ng/ml.

The present invention still further features in the described preferred invention the IGF is provided at a concentration of 20 ng/ml.

The present invention still further features in the described preferred invention the KGF is provided at the concentration of 4 to 100 ng/ml.

The present invention still further features in the described preferred invention the KGF is provided at a concentration of 20 ng/ml.

The present invention still further features in the described preferred invention the cells of the species mesenchymal stem cells maintain a doubling time of at least 20 to 28 hours.

The present invention still further features in the described preferred invention the human mesenchymal stem cells are maintainable in an undifferentiated and proliferative state for at least passage 40.

The present invention provides isolated mesenchymal stem cells that are surface antigen negative for CD10, CD13, CD14, CD29, CD34, CD45, and HLA Class I and II and are positive for CD 105, CD73, CD90, CD44, oct3/4 mRNA. In particular, the cell are surface antigen negative for CD3, CD14, CD19, CD31, CD34, CD36, CD38, CD45, CD62E and HLA-DR, Muc18, cKit, Tie/Tek, HLA-class I and 2-microglobulin and is positive for CD44, CD73, CD90, CD105, CD106, and Stro-1. The present invention provides an isolated multipotent non-embryonic, non-germ cell line cell that expresses transcription factors oct3/4 and does not express REX-1 and TERT.

The cells of the present invention described above may have the capacity to be induced to differentiate to form at least one differentiated cell type of mesodermal, ectodermal and endodermal origin. For example, the cells may have the capacity to be induced to differentiate to form cells of at least osteoblast, chondrocyte, adipocyte, fibroblast, marrow stroma, skeletal muscle, smooth muscle, cardiac muscle, endothelial, epithelial, hematopoietic, glial, neuronal or oligodendrocyte cell type.

The present disclosure provides a method of isolation and culturing mesenchymal stem cells (MSC) in an undifferentiated state for several passages.

Isolation and method for promoting mesenchymal stem cells (MSC) expansion while maintaining its pluripotency and its cryopreservation as a ready to use stem cells product for stem cell therapy are provided. The composition includes the improved method of isolation of mesenchymal stem cells using cocktail of antibodies. The composition also includes a xeno-free cell culture systems and large scale production in the cell factory for MSC expansion that comprise a xeno-free cell culture medium comprising a mixture of soluble MSC growth promoting factors. Compositions further include at least one or combination of protein supplement from human origin added in the culture medium. In the xeno-free culture system of present disclosure at least one or combinations of growth factor is added to support the growth of the cells and suitable for MSC expansion while maintaining pluripotency of the cells for at least 50 passages specifically 30 passages. The object of the present disclosure therefore is to provide improved culture system for isolation and expansion of pure population of human mesenchymal stem cells under xeno-free conditions.

Further, the present disclosure provides process of obtaining MSCs, the process comprise the use of the xeno-free cell culture systems and expanded MSCs as a ready to use product for cell transplantation to treat various disorders such as cardiovascular disorders, nervous system disorders, bone, cartilage and muscle disorders, diabetes and bone marrow disorders and autoimmune diseases.

The present disclosure provides a process of isolating and promoting proliferation of mesenchymal stem cells (MSCs) in large scale in the cell factory while maintaining pluripotency of the MSCs.

The present disclosure provides ready to use stem cells product for stem cell therapy by using process of cryopreservation. The composition includes the improved process for isolation of mesenchymal stem cells from either human bone marrow using cocktail of antibodies for negative selection of MSCs. The composition also includes a xeno-free cell culture systems and large scale production in the cell factory for MSCs expansion that comprise a xeno-free cell culture medium and 6230 sq cm cell culture surface area. The xeno-free culture medium is a solution that comprises a mixture of soluble MSCs growth promoting factors. The compositions further include at least one or combination of protein supplement from human origin is added in the culture medium. In the xeno-free culture system of present disclosure at least one or combinations of growth factor is added to support the growth of the cells and suitable for MSCs expansion while maintaining multipotency of the cells for at least 40 passages. The object of the present disclosure therefore is to provide improved culture system for isolation and expansion of pure population of human mesenchymal stem cells under xeno-free conditions.

The present disclosure provides a process of obtaining the MSCs by using a cocktail of antibodies to enrich MSCs and use of the xeno-free cell culture system to promote the expansion of MSCs in large quantity in cell factory. Further methods comprise use of the xeno-free cell culture systems and expanded MSCs as a ready to use product for cell transplantation to treat various disorders such as cardiovascular disorders, nervous system disorders, bone, cartilage and muscle disorders, diabetes and bone marrow disorders and autoimmune diseases.

The present invention addresses the shortcomings of the presently known configurations by providing cell culture system and methods for propagating and maintaining stem cells in an undifferentiated state and in an animal-free environment.

Surprisingly it was found that the culture medium disclosed in the present invention for growing or culturing, proliferating and maintaining the mesenchymal stem cells maintains the stability of the mesenchymal stem cell up to more than 30 passages preferably 40 passages, more preferably 35 passages, most preferably 30 passages. Further, methods for isolating, culturing, propagating and/or maintaining the mesenchymal stem cells provided in the present invention result in the stability of the mesenchymal stem cell up to more than 30 passages preferably 40 passages, more preferably 35 passages, most preferably 30 passages.

One embodiment of the present invention relates to isolation of bone marrow derived mesenchymal stem cells.

Another embodiment of the present invention relates to culture and propagation of bone marrow derived mesenchymal stem cells.

Yet another embodiment of the present invention relates to characterization of mesenchymal stem cells.

The present invention relates to novel method for isolation and culturing human mesenchymal stem cells in defined culture conditions. In the present invention mesenchymal stem cells are isolated from bone marrow and corneal epithelial tissue and are in pure population. In particular the present inventions relate to the improved systems for culturing human mesenchymal stem cells under xeno-free condition and scale-up it on a larger batch for human therapy. The defined culture supports the stem cell in a long term in vitro culture systems. The system described in this invention allows for proliferation of stem cells for use in studying the biology of stem cell differentiation, and the production of important products for use in human therapy.

In one embodiment, the present invention relates to the novel approach for isolation and culture of mesenchymal stem cells

In accordance with the present invention, one embodiment provides a process for proliferation of mesenchymal stem cells (MSCs), the process comprising;
obtaining mesenchymal stem cells from a sample which are positive for a marker selected from the group consisting of CD44, CD50, CD54, CD73, CD90, CD105, CD106, CD117, oct 3/4, Actin filament associated protein (AFAP), Frizzled7 (FZD7), Dickkopf3 (DKK3), Protein tyrosine phosphatase receptor F (PTPRF), RAB3B, and Stro-1;
culturing said mesenchymal stem cells in a growth medium selected from the group consisting of i) a culture medium comprising a basal medium selected from the group consisting of KO-DMEM, DMEM-LG and DMEM-F12 (1:1) or a combination thereof; 5-10% fetal bovine serum (FBS), growth factors, 200 mM GlutaMax™ and antibiotics; ii) a culture medium comprising basal medium selected from the group consisting of KO-DMEM, DMEM-LG and DMEM-F12 (1:1) or a combination thereof; growth factors, human serum, 200 mM GlutaMax™ and antibiotics; iii) a culture medium comprising basal medium selected from the group consisting of KO-DMEM, DMEM-LG and DMEM-F12 (1:1) or a combination thereof; growth factors, human plasma, heparin, 200 mM GlutaMax™ and antibiotics; and iv) a culture medium comprising basal medium selected from the group consisting of KO-DMEM, DMEM-LG and DMEM-F12 (1:1) or a combination thereof; growth factors, human plasma or human serum or a combination thereof, heparin, 200 mM GlutaMax™ and antibiotics.

In one embodiment, there is provided a process of isolation of mesenchymal stem cells (MSC) from a sample, the process comprising:
treating the sample with an antibody against an antigen, wherein said antigen is selected from a group consisting of CD44, CD50, CD54, CD73, CD90, CD105, CD106, CD117, oct 3/4, Actin filament associated protein (AFAP), Frizzled7 (FZD7), Dickkopf3 (MO), Protein tyrosine phosphatase receptor F (PTPRF), RAB3B, and Stro-1;
obtaining cells expressing said antigen; wherein said cells are mesenchymal stem cells; and
culturing said mesenchymal stem cells in a growth medium selected from the group consisting of i) a culture medium comprising a basal medium selected from the group consisting of KO-DMEM, DMEM-LG and DMEM-F12 (1:1) or a combination thereof; 5-10% fetal bovine serum (FBS), growth factors, 200 mM GlutaMax™ and antibiotics; ii) a culture medium comprising basal medium selected from the group consisting of KO-DMEM, DMEM-LG and DMEM-F12 (1:1) or a combination thereof; growth factors, human serum, 200 mM GlutaMax™ and antibiotics; iii) a culture medium comprising basal medium selected from the group consisting of KO-DMEM, DMEM-LG and DMEM-F12 (1:1) or a combination thereof; growth factors, human plasma, heparin, 200 mM GlutaMax™ and antibiotics; and iv) a culture medium comprising basal medium selected from the group consisting of KO-DMEM, DMEM-LG and DMEM-F12 (1:1) or a combination thereof; growth factors, human plasma or human serum or a combination thereof, heparin, 200 mM GlutaMax™ and antibiotics.

In another embodiment, the present invention provides a of isolation of mesenchymal stem cells (MSC) from a sample, wherein the process further comprises maintaining said mesenchymal stem cells after culturing in a maintenance medium selected from the group consisting of i) a culture medium comprising a basal medium selected from the group consisting of KO-DMEM, DMEM-LG and DMEM-F12 (1:1) or a combination thereof; 5-10% fetal bovine serum (FBS), growth factors, 200 mM GlutaMax™ and antibiotics; ii) a culture medium comprising basal medium selected from the group consisting of KO-DMEM, DMEM-LG and DMEM-F12 (1:1) or a combination thereof; growth factors, human serum, 200 mM glutamax and antibiotics; iii) a culture medium comprising basal medium selected from the group consisting of KO-DMEM, DMEM-LG and DMEM-F12 (1:1) or a combination thereof; growth factors, human plasma, heparin, 200 mM GlutaMax™ and antibiotics; and iv) a culture medium comprising basal medium selected from the group consisting of KO-DMEM, DMEM-LG and DMEM-F12 (1:1) or a combination thereof; growth factors, human plasma or human serum or a combination thereof, heparin, 200 mM GlutaMax™ and antibiotics.

One embodiment of the present invention is directed towards a process of for isolation of mesenchymal stem cells (MSCs), wherein the mesenchymal stem cells are isolated from a sample selected from the group consisting of bone marrow, corneal epithelial tissue, adipose tissue umbilical cord, placenta, dental ligament and dental pulp.

Another embodiment of the present invention is directed towards a process of for isolation of mesenchymal stem cells (MSCs), wherein the mesenchymal stem cells are isolated from bone marrow sample.

Another embodiment of the present invention is directed towards a process of for isolation of mesenchymal stem cells (MSCs), wherein the mesenchymal stem cells are isolated from corneal epithelial tissue.

Another embodiment of the present invention is directed towards a process of for isolation of mesenchymal stem cells (MSCs), wherein the mesenchymal stem cells are isolated from adipose tissue.

Another embodiment of the present invention is directed towards a process of for isolation of mesenchymal stem cells (MSCs), wherein the mesenchymal stem cells are isolated from umbilical cord.

Another embodiment of the present invention is directed towards a process of for isolation of mesenchymal stem cells (MSCs), wherein the mesenchymal stem cells are isolated from placenta.

Another embodiment of the present invention is directed towards a process of for isolation of mesenchymal stem cells (MSCs), wherein the mesenchymal stem cells are isolated from dental ligament.

Another embodiment of the present invention is directed towards a process of for isolation of mesenchymal stem cells (MSCs), wherein the mesenchymal stem cells are isolated from dental pulp.

Another embodiment of the present invention is directed towards a process of for isolation of mesenchymal stem cells (MSCs), wherein the mesenchymal stem cells are isolated from a sample obtained from human or murine origin.

In another embodiment of the present invention there is provided a growth medium; wherein the growth medium comprises a basal medium KO-DMEM, 5-10% fetal bovine serum (FBS), growth factors, 200 mM GlutaMax™ and antibiotics.

In yet another embodiment there are provided the growth factors; wherein said growth factors are selected from the group consisting of platelet derived growth factor (PDGF) 5 to 100 ng/ml, transforming growth factor-beta (TGF-beta) 5 to 50 ng/ml, keratinocyte growth factor (KGF) 4 to 100 ng/ml, basic Fibroblast growth factor (bFGF) 4 to 80 ng/ml, Epidermal growth factor (EGF) 5-50 ng/ml; 10 to 100000 U/ml Leukemia Inhibitory growth factor (LIF); 5 to 50 ng/ml and Insulin-like growth factor-I (IGF-I) 5 to 50 ng/ml.

In accordance with one aspect of the present invention, there is provided the concentration of the growth factors used in the culture medium and/or in maintenance medium of the mesenchymal stem cells, wherein the concentration of PDGF is 20 ng/ml.

In accordance with one aspect of the present invention the concentration of TGF which is 20 ng/ml.

In accordance with the second aspect of the present invention the concentration of KGF is a 20 ng/ml.

In accordance with the third aspect of the present invention the concentration of bFGF is 10 ng/ml.

In accordance with the fourth aspect of the present invention the concentration of EGF is 15 ng/ml.

In accordance with the fourth aspect of the present invention the concentration of IGF-I is 20 ng/ml.

In accordance with the fifth aspect of the present invention the concentration of human serum is in the range of 0.5 to 1%.

In accordance with the sixth aspect of the present invention the concentration of human plasma is in the range of 1 to 20%.

In accordance with the seventh aspect of the present invention the concentration of GlutaMax™ is 200 mM.

In accordance with the eighth aspect of the present invention the concentration of heparin is in the range of 1000 to 10,000 U/ml.

In accordance with the ninth aspect of the present invention the concentration of LIF is 10,000 U/ml.

In one embodiment, it is contemplated that the mesenchymal stem cells retain the functional characteristics without any abnormalities up to more than 30 passages.

In one embodiment, it is contemplated that the mesenchymal stem cells retain the functional characteristics without any abnormalities up to 40 passages.

In one embodiment, it is contemplated that the mesenchymal stem cells retain the functional characteristics without any abnormalities up to 32 passages.

In one embodiment, it is contemplated that the said mesenchymal stem cells retain the functional characteristics without any abnormalities up to 30 passages.

In one embodiment, it is contemplated that the mesenchymal stem cells retain the functional characteristics without any abnormalities till 25-30 passages.

In one embodiment, it is contemplated that the mesenchymal stem cells, retain the functional characteristics without any abnormalities till 30 passages.

In one embodiment, it is contemplated that the mesenchymal stem cells retain the functional characteristics without any abnormalities till 25 passages.

In another embodiment it is contemplated that the mesenchymal stem cells are capable of differentiation into cells selected from a group consisting of cardiac cells, neuronal cells, hepatocytes, pancreatic beta cells, osteoblasts, chondrocytes and adipocytes.

The mesenchymal stem cells of the present invention are negative for a marker selected from the group consisting of CD3, CD4, CD8, CD10, CD13, CD14, CD19, CD29, CD31, CD34, CD36, CD38, CD45, CD62E, CD66b, CD133, HLA I and II, HLA-DR, glycophorin-A, Muc 18, cKit, Tie/Tek, microglobulin, oct 4, Nanog, Rex-1, TDGF, TERT, SOX-2 and beta actin control.

In accordance with another aspect of the present invention, there is provided a cell culture medium for proliferation and/or maintenance of mesenchymal stem cells (MSCs), wherein said culture medium comprising KO-DMEM; growth factors selected from the group consisting of 5 to 100 ng/ml platelet derived growth factor (PDGF), 5 to 50 ng/ml transforming growth factor-beta (TGF-beta), 4 to 100 ng/ml keratinocyte growth factor (KGF), 4 to 80 ng/ml basic Fibroblast growth factor (bFGF), 5 to 50 ng/ml Epidermal growth factor (EGF); 10 to 100000 U/ml Leukemia Inhibitory growth factor (LIF) and 5 to 50 ng/ml Insulin-like growth factor-I (IGF-I); 5 to 10% (w/w) fetal bovine serum (FBS); 200 mM GlutaMax™; and antibiotics.

In accordance with another aspect of the present invention, there is provided a cell culture medium for proliferation and/or maintenance of mesenchymal stem cells (MSCs), wherein said culture medium comprising KO-DMEM; growth factors selected from the group consisting of 5 to 100 ng/ml platelet derived growth factor (PDGF), 5 to 50 ng/ml transforming growth factor-beta (TGF-beta), 4 to 100 ng/ml keratinocyte growth factor (KGF), 4 to 80 ng/ml basic Fibroblast growth factor (bFGF), 5 to 50 ng/ml Epidermal growth factor (EGF); 10 to 100000 U/ml Leukemia Inhibitory growth factor (LIF) and 5 to 50 ng/ml Insulin-like growth factor-I. (IGF-I); human plasma or human serum or a combination thereof; 200 mM GlutaMax™; and antibiotics.

In accordance with another aspect of the present invention, there is provided a cell culture medium for proliferation and/or maintenance of mesenchymal stem cells (MSCs), wherein said culture medium comprising basal medium selected from the group consisting of KO-DMEM, DMEM-LG and DMEM-F12 (1:1) or a combination thereof; growth factors selected from the group consisting of 5 to 100 ng/ml platelet derived growth factor (PDGF), 5 to 50 ng/ml transforming growth factor-beta (TGF-beta), 4 to 100 ng/ml keratinocyte growth factor (KGF), 4 to 80 ng/ml basic Fibroblast growth factor (bFGF), 5 to 50 ng/ml Epidermal growth factor (EGF); 10 to 100000 U/ml Leukemia Inhibitory growth factor (LIF) and 5 to 50 ng/ml Insulin-like growth factor-I (IGF-I); 0.5 to 1% human serum; 200 mM GlutaMax™; and antibiotics.

In accordance with another aspect of the present invention, there is provided a cell culture medium for proliferation and/or maintenance of mesenchymal stem cells (MSCs), wherein said culture medium comprising basal medium selected from the group consisting of KO-DMEM, DMEM-LG and DMEM-F12 (1:1) or a combination thereof; growth factors selected from the group consisting of 5 to 100 ng/ml platelet derived growth factor (PDGF), 5 to 50 ng/ml transforming growth factor-beta (TGF-beta), 4 to 100 ng/ml keratinocyte growth factor (KGF), 4 to 80 ng/ml basic Fibroblast growth factor (bFGF), 5 to 50 ng/ml Epidermal growth factor (EGF); 10 to 100000 U/ml Leukemia Inhibitory growth factor (LIF) and 5 to 50 ng/ml Insulin-like growth factor-I (IGF-I); 1 to 20% human plasma; 200 mM GlutaMax™; 1000 to 10,000 U/ml heparin and antibiotics.

In accordance with another aspect of the present invention, there is provided a cell culture medium for proliferation and/or maintenance of mesenchymal stem cells (MSCs), wherein said culture medium comprising basal medium selected from the group consisting of KO-DMEM, DMEM-LG and DMEM-F12 (1:1) or a combination thereof; growth factors selected from the group consisting of 5 to 100 ng/ml platelet derived growth factor (PDGF), 5 to 50 ng/ml transforming growth factor-beta (TGF-beta), 4 to 100 ng/ml keratinocyte growth factor (KGF), 4 to 80 ng/ml basic Fibroblast growth factor (bFGF), 5 to 50 ng/ml Epidermal growth factor (EGF); 10 to 100000 U/ml Leukemia Inhibitory growth factor (LIF) and 5 to 50 ng/ml Insulin-like growth factor-I (IGF-I); 0.5 to 1% human serum or 1 to 20% human plasma; 200 mM GlutaMax™; 1000 to 10,000 U/ml heparin and antibiotics.

The mesenchymal stem cells described herein were isolated by the method developed by the inventors, who identified a number of specific cell surface markers that characterize the mesenchymal stem cells (MSCs). The method of the present invention can be used to isolate multipotent adult stem cells from any adult, child, or fetus, of human, murine and other species origin. It is therefore now possible for one of skill in the art to obtain bone marrow aspirates, brain or liver biopsies, and possibly other organs, and isolate the cells using positive or negative selection techniques known to those of skill in the art, relying upon the surface markers expressed on these cells, as identified by the inventors, without undue experimentation.

In one embodiment bone marrow mononuclear cells were derived from bone marrow aspirates by standard means known to those of skill in the art to obtain the mesenchymal stem cells. The mesenchymal stem cells are present within the bone marrow (or other organs such as liver or brain) but do not express the common leukocyte antigen CD45 or erythroblast specific glycophorin-A (Gly-A). Negative selection is used to isolate cells using a combination of cell-specific markers identified by the inventors and described herein, such as cells are then subjected to using cocktail of antibody complexes directed against cell surface antigens of human hematopoietic cells and glycophorin A (Gly-A). The target Cells are easily collected as a purified population from Ficoll-plasma interface. The cells were immediately cultured in a defined medium.

The present inventions have shown that mesenchymal stem cells are maintained in Dulbecco Modified Essential Medium (DMEM) with low glucose or Knock-out Dulbeco Modified Essential Medium (Ko-DMEM) or DMEM-F12 supplemented with human serum, human plasma, heparin and growth factors.

The invention provides isolated cells cultured in a culture medium, which comprises of 0.5-5% human serum, 1-20% human plasma, 1000-10,000 U/ml heparin and growth factors selected from group of 5-20 ng/ml PDGF, 5-20 ng/ml TGF, 4-20 ng/ml KGF, 4-20 ng/ml FGF, 5-20 ng/ml EGF, 5-20 ng/ml IGF, and 100-1000 U/ml LIF or combination thereof growth factors use in the culture in vitro, purification and expansion of human mesenchymal stem cells useful for the elaboration of proper pharmaceutical compositions.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

### EXAMPLES

It should be understood that the following examples described herein are for illustrative purposes only and that various modifications or changes in light will be suggested to persons skilled in the art and are to be included within the spirit and purview of this application and the scope of the appended claims.

### Example 1

### Isolation of Mesenchymal Stem cells from bone marrow

Bone marrow sample (10-50 ml) of was aseptically aspirated from the iliac crest of the donors under deep sedation. The bone marrow was diluted (1:1) with Dulbecco's Modified Eagle's Medium (DMEM) Low Glucose (1000 mg/ml; DMEM-LG), Knock out DMEM (KO-DMEM), DMEM-F12. The bone marrow was centrifuged at 1200-1800 rpm for 10 minutes to remove anticoagulants. The supernatant was discarded and the bone marrow was washed once with the respective culture medium. Mononuclear cells (MNCs) were isolated by layering onto a Ficoll density gradient (1:2) (Stem Cell Technologies). The MNCs present in the buffy coat were washed again with respective culture medium. The mononuclear fraction containing MSCs were plated onto T-75 flasks (BD Biosciences) and cultured in various growth medium as provided in Table 1. All the media was supplemented with or without 10% FBS (Hyclone), 200 mM Glutamax (Invitrogen) and Pen-Strep (Invitrogen). The non-adherent cells were removed after 48 hours of culture and fresh medium was added. Subsequently medium was replenished every 48 hrs. FIGS. 1 (a) and (b) shows a monolayer of adult bone marrow derived mesenchymal stem cells under xeno-free condition.

TABLE 1
Medium composition for culturing, propagating
and maintaining the mesenchymal stem cells

| Medium No. | Medium Composition |
|---|---|
| Medium 1 | a basal medium KO-DMEM; 5-10% fetal bovine serum (FBS), |
| | growth factors, 200 mM GlutaMax ™ and antibiotics |
| Medium 2 | a basal medium DMEM; 5-10% fetal bovine serum (FBS), growth |
| | factors, 200 mM GlutaMax ™ and antibiotics |
| Medium 3 | a basal medium DMEM-F12 (1:1); 5-10% fetal bovine serum (FBS), |
| | growth factors, 200 mM GlutaMax ™ and antibiotics |
| Medium 4 | combination of KO-DMEM, DMEM-LG and DMEM-F12; 5-10% |
| | fetal bovine serum (FBS), growth factors, 200 mM GlutaMax ™ and |
| | antibiotics |
| Medium 5 | a basal medium selected from the group consisting of KO-DMEM, |
| | DMEM-LG and DMEM-F12 (1:1) or a combination thereof; growth |
| | factors, human serum, 200 mM GlutaMax ™ and antibiotics |
| Medium 6 | a basal medium selected from the group consisting of KO-DMEM, |
| | DMEM-LG and DMEM-F12 (1:1) or a combination thereof; growth |
| | factors, human plasma, heparin, 200 mM GlutaMax ™ and |
| | antibiotics |
| Medium 7 | a basal medium selected from the group consisting of KO-DMEM, |
| | DMEM-LG and DMEM-F12 (1:1) or a combination thereof; growth |
| | factors, human plasma or human serum or a combination thereof, |
| | heparin, 200 mM GlutaMax ™ and antibiotics. |

### Example 2

### Selecting the Mesenchymal Stem Cell by Negative Expression

Antibody cocktail solution (104 comprising CD3, CD4, CD8, CD14, CD19, CD38, and CD66b and glycophorin-A antibodies was directly added to 10 ml of fresh bone marrow samples and incubated for 20 minutes at room temperature. The unwanted cells form the antigen-antibody complex. This makes the cells heavier and hence settles with the red cell population at the bottom layer. Thus, an enriched population of MSCs at the Ficoll interface was obtained. The target cells are collected as a purified population from Ficoll interface.

### Example 3

### Propagation or Proliferation of Bone Marrow Derived Mesenchymal Stem Cells

Once the mesenchymal stem cells obtained from the process as described in Example 1 became confluent, they were dissociated with 0.25% trypsin/0.53 mM EDTA (Invitrogen) and re-seeded at the rate of 10,000 cells per cm² (passage 1). After 3-5 days of culture, the cells reached 90% confluency, and were sub-cultured in the culture medium having composition as provided in Table 1 for subsequent propagation.

### Example 4

### Maintenance of Mesenchymal Stem Cells

The proliferated mesenchymal stem cells as described in Example 3 were maintained in the medium as provided in Table 1.

### Example 5

### Characterization of Mesenchymal Stem Cells

### Characterization of MSCs by Flow Cytometry

The characterization of cell surface cluster differentiation (CD) markers on MSCs was performed to aid analysis of the expression of cell surface markers. Aliquots of MSCs were allowed to expand at 37° C. and 95% air/5% CO₂ humidified environment. After expansion, cells were dissociated with 0.25% trypsin-EDTA and re-suspended in buffer. The cells were then centrifuged and re-suspended in wash buffer at a concentration of 1×10⁶ cells/ml. Wash buffer consisted of phosphate buffer supplemented with 1% (v/v) FBS and 1% (w/v) sodium azide. Cell viability was >98% by the Trypan blue exclusion process. 100 μl of cell preparation 1×10⁶ were stained with saturating concentrations of fluorescein isothiocyanate-(FITC), phycoerythrin-(PE); conjugated markers and isotype matched controls. Briefly, cells were incubated in the dark for 30 min. at 4° C. After incubation, cells were washed three times with wash buffer and resuspended in 0.5 ml of wash buffer for analysis on the flow cytometer. Flow cytometry was performed on a LSR-II. Cells were identified by light scatter. Logarithmic fluorescence was evaluated (4 decade, 1024 channel scale) on 10,000 gated events. Analysis was performed using FACS DIVA software and the presence or absence of each antigen was determined by comparison to the appropriate isotype control. An antigenic event was observed when the fluorescence was greater than 25% above its isotype control. Statistical analysis was performed on the pooled flow cytometric data from the three mesenchymal stem cell lines. Thus, a sample size of three was used for each CD marker. A mean value above 10,000 cells was considered positive for any CD marker.

Flow cytometry showed cell populations positive for CD44, CD50, CD54, CD73, CD90, CD105, CD106 and CD117; and negative for CD3, CD4, CD8, CD10, CD13, CD14, CD19, CD29, CD31, CD34, CD36, CD38, CD45, CD62E, CD66b and CD133.

### Characterization of MSCs for Pluripotent Markers by RT-PCR

MSCs were analyzed for the pluripotent markers after passage 4 by RT-PCR.

RNA extractions were carried out with the RNeasy mini kit. MSCs were vortexed for 1 min to shear genomic DNA before loading onto the columns, and then eluted in a minimum volume of 30 μl and a maximum volume of 2×50 μl RNAse-free water. RNA obtained with this procedure was essentially free of genomic DNA. When using different extraction procedures, a DNAse I treatment, followed by phenol extraction and ethanol precipitation, was applied to remove traces of contaminating DNA.

RNA obtained from the cells was reverse transcribed in the presence of 5 mM MgCl₂, 1×PCR Buffer II, 1 mM dNTPs, 25u MuLV Reverse Transcriptase, 1u RNA inhibitor, 2.5 μM Random hexamers in a final reaction volume of 20 Reactions were carried out at 42° C. for 30 minutes in a thermocycler, followed by a 10 minute step at 99° C., and then by cooling to 4° C.

cDNA (2 μl) of products were amplified with 1 unit of Taq polymerase in the buffer provided by the manufacturer which contains no MgCl₂, and in the presence of the specific primers having nucleotide sequence as shown in table together with the beta-actin primers (SEQ ID NO:10 and 20) used as an internal control. The amount of dNTPs carried over from the reverse transcription reaction is fully sufficient for further amplification. A first cycle of 10 minutes at 95° C., 45 seconds at 65° C. and 1 minute at 72° C. was followed by 45 seconds at 95° C., 45 seconds at 65° C. and 1 minute at 72° C. for 30 cycles. The conditions were chosen so that none of the RNAs analyzed reached a plateau at the end of the amplification protocol, i.e. they were in the exponential phase of amplification, and that the two sets of primers used in each reaction did not compete with each other. Each set of reactions always included a no-sample negative control.

The PCR products were loaded onto ethidium bromide stained 1 to 2% (depending on the size of the amplification products) agarose gels in TBE. A 100 by DNA ladder molecular weight marker was run on every gel to confirm expected molecular weight of the amplification product.

Images of the RT-PCR ethidium bromide-stained agarose gels were acquired with a gel documentation system and quantification of the bands was performed. Band intensity was expressed as relative absorbance units. The ratio between the sample RNA to be determined and GAPDH or Actin was calculated to normalize for initial variations in sample concentration and as a control for reaction efficiency. Mean and standard deviation of all experiments performed were calculated after normalization to beta-Actin.

RT-PCR analysis showed that MSCs are negative for the pluripotent markers viz. OCT-4, Nanog, Rex-1, TDGF, TERT, and SOX-2. The primer sequences of the pluripotent markers and the results are provided in Table 2 and 3.

TABLE 2
Primer sequences

| | SEQ | | No of | |
|---|---|---|---|---|
| | ID | | nucleo- | Re- |
| Primers | NO | Sequence | tide | sult |
| OCT-4 | 1 | CGACCATCTGCCGCTTTGAG | 572 | −ve |
| | 2 | CCCCCTGTCCCCCATTCCTA | | |
| Nanog | 3 | CCTCCTCCATGGATCTGCTTATTCA | 262 | −ve |
| | 4 | CAGGTCTTCACCTGTTTGTAGCTGAG | | |
| Rex-1 | 5 | GCGTACGCAAATTAAAGTCCAGA | 303 | −ve |
| | 6 | CAGCATCCTAAACAGCTCGCAGAAT | | |
| TDGF1 | 7 | GCCCGCTTCTCTTACAGTGTGATT | 498 | −ve |
| | 8 | AGTACGTGCAGACGGTGGTAGTTCT | | |
| SOX2 | 9 | CCCCCGGCGGCAATAGCA | 448 | −ve |
| | 10 | TCGGCGCCGGGGAGATACAT | | |
| TERT | 13 | AGCTATGCCCGGACCTCCAT | 602 | −ve |
| | 14 | GCCTGCAGCAGGAGGATCTT | | |
| Beta- | 19 | GCTCGTCGTCGACAACGGCT | 353 | +ve |
| actin | 20 | CAAACATGATCTGGGTCATCTTCTC | | con- |
| | | | | trol |

TABLE 3
Analysis of Pluripotent Markers on MSCs by RT PCR

| Pluripotent/Stemness Markers | MSCs |
|---|---|
| Oct4 | −ve |
| Nanog | −ve |
| Sox2 | −ve |
| Rex1 | −ve |
| TDGF1 | −ve |

### Characterization of MSCs for Multipotent Markers by RT-PCR

Cells were analyzed for the multipotent markers after passage 4. MSCs were analyzed for the expression of multipotent markers by RT-PCR. MSCs were positive for Actin filament associated protein (AFP), Frizzled7 (FZD7), Dickkopf3 (DKK3) and Protein tyrosine phosphatase receptor F (PTPRF) (Table 4).

RNA was isolated as described as above and RT PCR was carried out with the primer sequences of the multipotent markers. The primer sequences can be designed from the known nucleotide sequence of these markers.

TABLE 4
analysis of multipotent markers on MSCs by RT-PCR

| Multipotent/Stemness Markers | MSCs |
|---|---|
| β-actin control | +ve |
| Actin filament associated protein (AFAP) | +ve |
| Frizzled7 (FZD7) | +ve |
| Dickkopf3 (DKK3) | +ve |
| Protein tyrosine phosphatase receptor F (PTPRF) | +ve |
| RAB3B | +ve |

### Example 6

### Differentiation of Mesenchymal Stem Cells

### Osteogenic Differentiation

Isolated mesenchymal stem cells as described above were plated at low density on tissue-culture-treated dishes in the presence of 10 mM b-glycerol phosphate, 0.1 M dexamethasone, and 200 μM ascorbic acid in α-MEM medium with or without 10% FBS for 3-4 week

DMEM supplemented with or without 10% FBS (Hyclone), 200 mM GlutaMax™ (Invitrogen), 10⁻⁸ dexamethasone (Sigma-Aldrich), 30 μg/ml ascorbic acid (Sigma-Aldrich) and 10 mM β-glycerophosphate (Sigma-Aldrich) for 3 weeks (Phinney et al., 1999). Fresh medium was replenished every 3 days. Calcium accumulation was assessed by Von Kossa Staining. The differentiated cells were washed with PBS and fixed with 10% formalin for 30 minutes. The fixed cells were incubated with 5% AgNO₃ for 60 minutes under UV light and then treated with 2.5% sodium thiosulphate for 5 minutes. Images were captured using Nikon Eclipse 90i microscope (Nikon Corporation) and Image-Pro Express software (Media Cybernetics, Inc, Silver Spring).

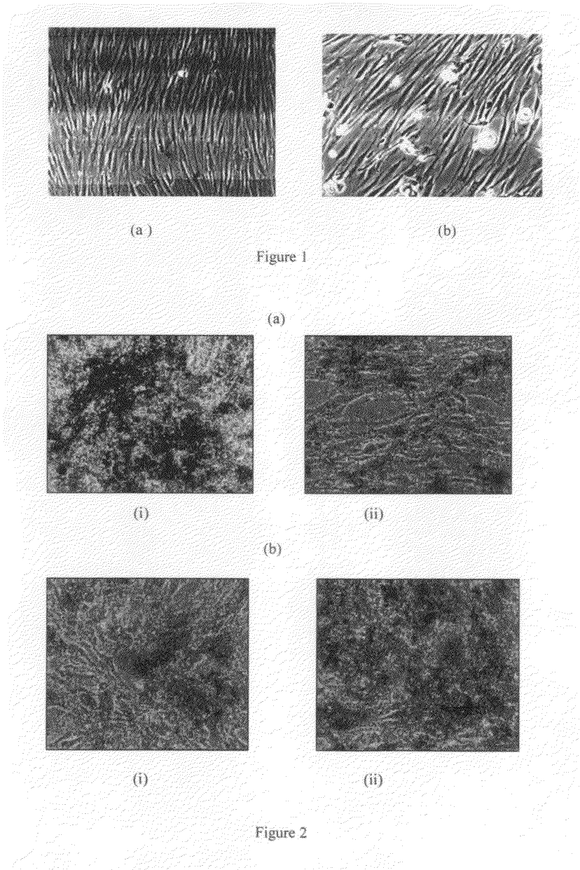

The mesenchymal stem cells were differentiated into osteoblast cell. FIG. 2(a) shows differentiation of the mesenchymal stem cells in osteoblast cells at passage 5 and FIG. 2(b) shows differentiation of the mesenchymal stem cells in osteoblast cells at passage 25.

### Adipogenic Differentiation

Mesenchymal stem cells were grown to confluence followed by exposure to 1 mM dexamethasone, 10 μg/ml insulin, and 0.5 mM isobutylxanthine in α-MEM medium containing 10% FBS for 2 to 4 weeks.

Human MSCs were cultured for up to 3 weeks in DMEM supplemented with 10% FBS, 200 mM GlutaMax™, 1 μM dexamethasone, isobutylmethylxanthine, 1 μg/ml insulin and 100 μM indomethacin (all Sigma-Aldrich). Medium with inducing factors was replenished every 3 days. Cells were fixed in 10% formalin for 20 minutes. 200 μl of Oil Red 0 staining solution was added and incubated for 10 minutes at room temperature. The cells were rinsed five times with distilled water. The dye retained by the cells was eluted by incubation with 750 μl of isopropanol (Merck) and images were captured using Nikon Eclipse 90i microscope (Nikon) and Image-Pro Express software (Media Cybernetics).

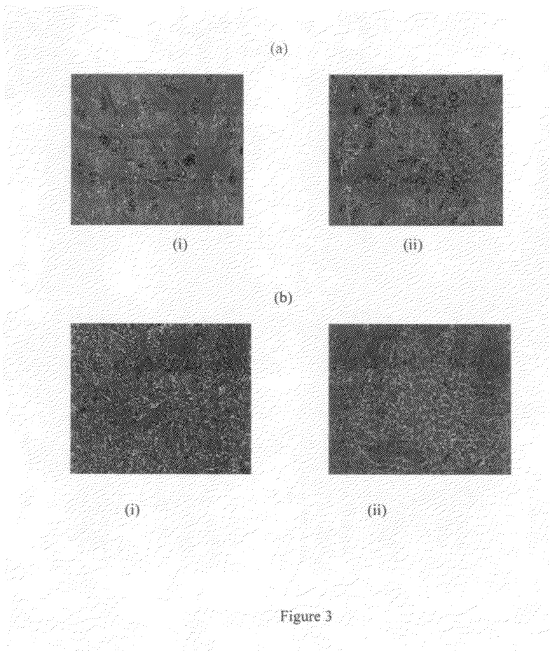

The mesenchymal stem cells were differentiated into adipocyte cell. FIG. 3(a) shows differentiation of the mesenchymal stem cells in adipocyte cells at passage 5 and FIG. 2(b) shows differentiation of the mesenchymal stem cells in adipocyte cells at passage 25.

### Example 7

### Karyotyping

It has been reported that karyotype instability can sometimes be observed with long-term passages of cells. In order to determine the karyotypic instability, karyotyping of the Human embryonic stem cell was done after every 10 passages. Human ES cells were grown in 60 mm plate on high density. Colcemid solution was added on the following day directly into the plate at the final concentration of 0.02 μg/ml. Cells were incubated for 2 hours at 37° C. and 5% CO₂. Culture media containing colcemid was removed after the incubation was over and cells were dissociated with 0.05% trypsin free from EDTA. Cells were transferred into 15 ml tube and 10 ml FBS in DMEM-F-12 was added. Cells were washed by centrifuging at 1000 rpm for 5 minutes at room temperature. Supernatant was removed and re-suspend the pellet in 2 ml of warm hypotonic solution. Cells were mixed properly and incubated in a water bath at 37° C. for 30 minutes. 0.5 ml of fixative is added drop-wise with swirling. Cells were centrifuged again at 1000 rpm for 5 minutes at room temperature. Supernatant was aspirated and 1 ml of fixative was added drop-wise while swirling the cells. This was done at least 2 times.

To make the spread, surface of the slide is humidified by application of warm breath whilst holding the slide at a 45° angle. One drop of the suspended cells is carefully dropped from the height of approximately 0.5 meter using Pasteur pipette onto the top surface of the slide and it was allowed to air dry.

Slide was stained with freshly made Leishman's stain for 8 minutes and was rinsed in running water for 1 minute and air dried. Cells were mounted with coverslip using depex.

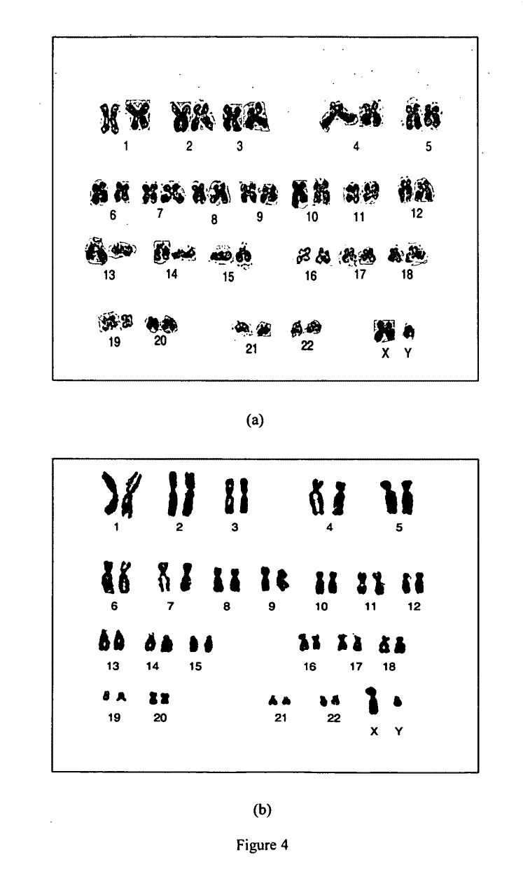

The karyotypes depicted in FIG. 4 shows the stability of the mesenchymal stem cells disclosed in the present invention at passage 25 confirming the stability of the MSCs of the present invention. The cells also showed the stability till passage 30.

All publications, patents and patent applications are incorporated herein by reference.

We claim:

1. A process of isolation of mesenchymal stem cells (MSC) from a sample, said process comprising;
a) treating said sample with an antibody against an antigen, wherein said antigen is selected from a group consisting of CD44, CD50, CD54, CD73, CD90, CD105, CD106, CD117, oct 3/4, Actin filament associated protein (AFAP), Frizzled7 (FZD7); Dickkopf3 (DKK3), Protein tyrosine phosphatase receptor F (PTPRF), RAB3B, and Stro-1;
b) obtaining cells expressing said antigen; wherein said cells are mesenchymal stem cells; and
c) culturing said mesenchymal stem cells in a growth medium selected from the group consisting of i) a culture medium comprising a basal medium selected from the group consisting of KO-DMEM, DMEM-LG and DMEM-F12 (1:1) or a combination thereof; 5-10% fetal bovine serum (FBS), growth factors, 200 mM GlutaMax™ and antibiotics; ii) a culture medium comprising basal medium selected from the group consisting of KO-DMEM, DMEM-LG and DMEM-F12 (1:1) or a combination thereof; growth factors, human serum, 200 mM GlutaMax™ and antibiotics; iii) a culture medium comprising basal medium selected from the group consisting of KO-DMEM, DMEM-LG and DMEM-F12 (1:1) or a combination thereof; growth factors, human plasma, heparin, 200 mM GlutaMax™ and antibiotics; and iv) a culture medium comprising basal medium selected from the group consisting of KO-DMEM, DMEM-LG and DMEM-F12 (1:1) or a combination thereof; growth factors, human plasma or human serum or a combination thereof, heparin, 200 mM GlutaMax™ and antibiotics.

2. The process as claimed in claim 1, wherein said process further comprises maintaining said mesenchymal stem cells after culturing as in step (c) of claim 1 in a maintenance medium selected from the group consisting of i) a culture medium comprising a basal medium selected from the group consisting of KO-DMEM, DMEM-LG and DMEM-F12 (1:1) or a combination thereof; 5-10% fetal bovine serum (FBS), growth factors, 200 mM GlutaMax™ and antibiotics; ii) a culture medium comprising basal medium selected from the group consisting of KO-DMEM, DMEM-LG and DMEM-F12 (1:1) or a combination thereof; growth factors, human serum, 200 mM GlutaMax™ and antibiotics; iii) a culture medium comprising basal medium selected from the group consisting of KO-DMEM, DMEM-LG and DMEM-F12 (1:1) or a combination thereof; growth factors, human plasma, heparin, 200 mM GlutaMax™ and antibiotics; and iv) a culture medium comprising basal medium selected from the group consisting of KO-DMEM, DMEM-LG and DMEM-F12 (1:1) or a combination thereof; growth factors, human plasma or human serum or a combination thereof, heparin, 200 mM glutamax and antibiotics.

3. The process as claimed in claim 1, wherein said sample is selected from the group consisting of bone marrow, corneal epithelial tissue, adipose tissue, umbilical cord, placenta, dental ligament and dental pulp.

4. The process as claimed in claim 1, wherein said sample is obtained from human or murine origin.

5. The process as claimed in claim 1, wherein said growth medium comprises a basal medium KO-DMEM, 5-10% fetal bovine serum (FBS), growth factors, 200 mM GlutaMax™ and antibiotics.

6. The process as claimed in claim 1, wherein said growth factors are selected from the group consisting of platelet derived growth factor (PDGF) 5 to 100 ng/ml, transforming growth factor-beta (TGF-beta) 5 to 50 ng keratinocyte growth factor (KGF) 4 to 100 ng/ml, basic Fibroblast growth factor (bFGF) 4 to 80 ng/ml, Epidermal growth factor (EGF) 5 to 50 ng/ml and Insulin-like growth factor-I (IGF-I) 5 to 50 ng/ml.

7. The process as claimed in claim 1, wherein concentration of PDGF 20 ng/ml.

8. The process as claimed in claim 1, wherein concentration of TGF is 20 ng/ml.

9. The process as claimed in claim 1, wherein concentration of said KGF is a 20 ng/ml.

10. The process as claimed in claim 1, wherein concentration of bFGF is 10 ng/ml.

11. The process as claimed in claim 1, wherein concentration of EGF is 15 ng/ml.

12. The process as claimed in claim 1, wherein concentration of IGF-I is 20 ng/ml.

13. The process as claimed in claim 1, wherein concentration of human serum is in the range of 0.5 to 1% (w/w).

14. The process as claimed in claim 1, wherein concentration of human plasma is in the range of 1 to 20% (w/w).

15. The process as claimed in claim 1, wherein concentration of GlutaMax™ is 200 mM.

16. The process as claimed in claim 1, wherein concentration of heparin is in the range of 1000 to 10,000 U/ml.

17. The process as claimed in claim 1, wherein said MSCs are negative for a marker selected from the group consisting of CD3, CD4, CD8, CD10, CD13, CD14, CD19, CD29, CD31, CD34, CD36, CD38, CD45, CD62E, CD66b, CD133, HLA I and II, HLA-DR, glycophorin-A, Muc18, cKit, Tie/Tek, microglobulin, oct 4, Nanog, Rex-1, TDGF, TERT, SOX-2 and beta actin control.

18. The process as claimed in claim 1, wherein said mesenchymal stem cells retain the functional characteristics without any abnormalities up to more than 30 passages.

19. The process as claimed in claim 1, wherein said mesenchymal stem cells retain the functional characteristics without any abnormalities up to 30 passages.

20. The process as claimed in claim 1, wherein said mesenchymal stem cells are capable of differentiation into cells selected from a group consisting of cardiac cells, neuronal cells, hepatocytes, pancreatic beta cells, osteoblasts, chondrocytes and adipocytes.

21. A cell culture medium for proliferation and/or maintenance of mesenchymal stem cells (MSCs), wherein said culture medium comprising KO-DMEM; growth factors selected from the group consisting of 5 to 100 ng/ml platelet derived growth factor (PDGF), 5 to 50 ng/ml transforming growth factor-beta (TGF-beta), 4 to 100 ng/ml keratinocyte growth factor (KGF), 4 to 80 ng/ml basic Fibroblast growth factor (bFGF), 5 to 50 ng/ml Epidermal growth factor (EGF) and 5 to 50 ng/ml Insulin-like growth factor-I (IGF-I); 5 to 10% (w/w) fetal bovine serum (FBS); 200 mM GlutaMax™; and antibiotics.

22. A cell culture medium for proliferation and/or maintenance of mesenchymal stem cells (MSCs), wherein said culture medium comprising basal medium selected from the group consisting of KO-DMEM, DMEM-LG and DMEM-F12 (1:1) or a combination thereof; growth factors selected from the group consisting of 5 to 100 ng/ml platelet derived growth factor (PDGF), 5 to 50 ng/ml transforming growth factor-beta (TGF-beta), 4 to 100 ng/ml keratinocyte growth factor (KGF), 4 to 80 ng/ml basic Fibroblast growth factor (bFGF), 5 to 50 ng/ml Epidermal growth factor (EGF) and 5 to 50 ng/ml Insulin-like growth factor-I (IGF-I); 0.5 to 1% human serum or 1 to 20% human plasma; 200 mM GlutaMax™; 1000 to 10,000 U/ml heparin and antibiotics.
